(19) United States
(12) Patent Application Publication
(10) Pub. No.: US 2024/0000183 A1
(43) Pub. Date: Jan. 4, 2024

(54) FOOTWEAR AND ACTION DETERMINATIONSYSTEM

(71) Applicant: SINTOKOGIO, LTD, Nagoya-shi (JP)

(72) Inventors: Yoshikane TANAAMI, Nagoya-shi (JP); Miyuki HAYASHI, Nagoya-shi (JP)

(73) Assignee: SINTOKOGIO, LTD, Nagoya-shi (JP)

(21) Appl. No.: 18/325,270

(22) Filed: May 30, 2023

(30) Foreign Application Priority Data
Jun. 30, 2022 (JP) 2022-106272

(51) Int. Cl. A43B 5/00; A43B 1/05; A43B 3/48; A43B 3/44

(52) U.S. Cl. CPC A43B 5/00; A43B 1/05; A43B 3/48; A43B 3/44

(57) ABSTRACT

Footwear includes a capacitive sensor, and conductive threads are woven into a fabric of the footwear, the conductive threads serving as the capacitive sensor configured to detect, on the basis of a change in capacitance between the conductive threads adjacent to each other, a change in pressure applied to an area into which the conductive threads are woven.

This Nonprovisional application claims priority under U.S.C. § 119 on Patent Application No. 2022-106272 filed in Japan on Jun. 30, 2022, the entire contents of which are hereby incorporated by reference.

### TECHNICAL FIELD

The present invention relates to footwear and an action determination system.

### BACKGROUND ART

For example, as disclosed in Patent Literature 1, a technique has been known of sewing a conductive woven fabric into cloth such as a sock to achieve a capacitive sensor configured to detect a change in electric capacity occurring between a human body and the conductive woven fabric.

### CITATION LIST

### Patent Literature

[Patent Literature 1]
Published Japanese Translation of PCT International Application Tokuhyo No. 2013-534833

### SUMMARY OF INVENTION

### Technical Problem

However, Patent Literature 1 does not disclose the configuration in which a change in pressure is detected on the basis of a change in capacitance caused by increasing proximity between adjacent two of conductive threads which are woven into a fabric.

It is an object of an aspect of the present invention to provide footwear and an action determination system that each have a novel configuration and can detect a change in pressure.

### Solution to Problem

In order to solve the foregoing problem, footwear in accordance with an aspect of the present invention includes a capacitive sensor. Into a fabric of the footwear, conductive threads are woven. The conductive threads serve as a capacitive sensor configured to detect, on the basis of a change in capacitance between the conductive threads adjacent to each other, a change in pressure applied to an area into which the conductive threads are woven.

Further, in order to solve the foregoing problem, an action determination system in accordance with an aspect of the present invention includes footwear, an acceleration sensor, and a controller. The footwear includes a capacitive sensor. The acceleration sensor is a sensor configured to detect a movement of an upper limb of a user and is worn around the upper limb of the user. The controller is configured to execute a determination process of determining an action of a whole body of the user. The determination process is executed on the basis of a change in pressure detected by the sensor of the footwear and a change in acceleration of the upper limb of the user detected by the acceleration sensor.

### Advantageous Effects of Invention

According to an aspect of the present invention, it is possible to provide footwear and an action determination system which each have a novel configuration and can detect a change in pressure.

### BRIEF DESCRIPTION OF DRAWINGS

FIG. 1 is a view schematically illustrating a configuration of a room boot in accordance with Embodiment 1 of the present disclosure.

FIG. 2 is a block diagram illustrating a configuration of an action determination system in accordance with Embodiment 2 of the present disclosure.

FIG. 3 is a view schematically illustrating the action determination system illustrated in FIG. 2.

### DESCRIPTION OF EMBODIMENTS

### Outlines of Embodiments of the Present Disclosure

First, the following will describe outlines of embodiments of the present disclosure.

### (Item 1)

Footwear including a capacitive sensor, wherein:
conductive threads are woven into a fabric of the footwear; and
the conductive threads serve as the capacitive sensor configured to detect, on the basis of a change in capacitance between the conductive threads adjacent to each other, a change in pressure applied to an area into which the conductive threads are woven.

### (Item 2)

The footwear according to Item 1, wherein the conductive threads are woven into a position corresponding to a portion to which a pressure is to be applied in a foot of a user and which is in an area in contact with at least one selected from the group consisting of: a toe of the user; a sole of the user; a heel of the user; and a dorsal surface of the foot of the user.

### (Item 3)

The footwear according to Item 1 or 2, further including a transmitting section configured to transmit, to an external device, data indicating a change in pressure detected by the capacitive sensor,
the transmitting section being detachably attached to the footwear.

### (Item 4)

An action determination system including:
the footwear according to any one of Items 1 to 3;
an acceleration sensor worn around an upper limb of a user and configured to detect a movement of the upper limb of the user; and
a controller configured to execute a determination process of determining an action of a whole body of the user on a basis of a change in pressure detected by the capacitive sensor of the footwear and a change in acceleration of the upper limb of the user detected by the acceleration sensor.

### Examples of Embodiments of the Present Disclosure

The following will describe embodiments of the present disclosure in detail with reference to the drawings. In the descriptions of the drawings, the same components are assigned the same reference signs, and the same descriptions are omitted.

In the following descriptions of embodiments, a basketball game is taken as an example.

### Embodiment 1

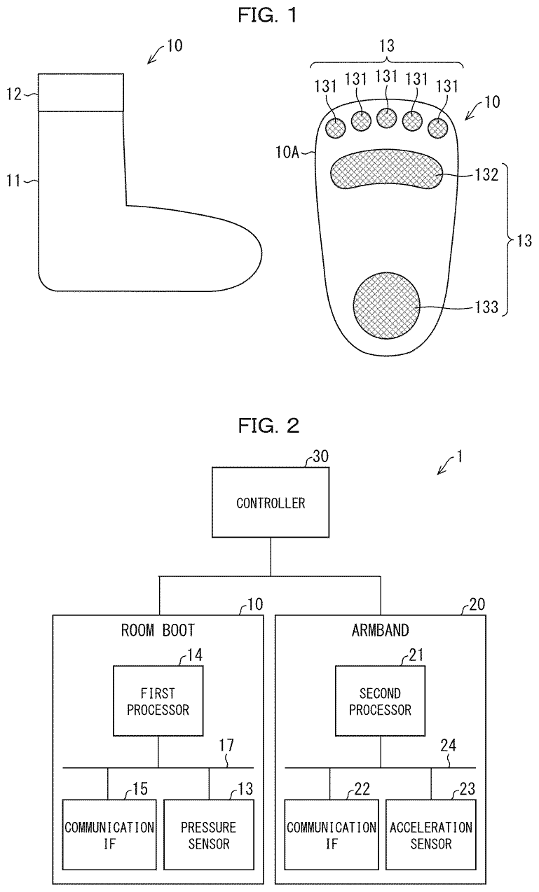

The following will describe footwear in accordance with Embodiment 1 of the present disclosure with reference to FIG. 1. FIG. 1 is a view schematically illustrating a configuration of a room boot 10.

<Room Boot>

The room boot 10 is one example of footwear worn indoors. The room boot 10 detects a movement of a foot of a user Us. In Embodiment 1, the room boot 10 is a shoe for indoor use. The room boot 10 performs wireless communications with an external device. Note that the room boot 10 may perform wired communications with an external device.

As illustrated in FIG. 1, the room boot 10 is constituted by a boot main body 11 and a transmitting section 12. Into a fabric of the boot main body 11, conductive threads are woven. The conductive threads serve as a pressure sensor 13 configured to detect, on the basis of a change in a capacitance between the conductive threads adjacent to each other, a change in pressure applied to an area into which the conductive threads are woven. The pressure sensor 13 is a capacitive sensor. Application of a pressure to the boot main body 11 causes deformation of the boot main body 11 and causes the conductive threads adjacent to each other to approach each other. The approach of the conductive threads adjacent to each other causes a change in a capacitance between the conductive threads. The pressure sensor 13 detects the change in the capacitance to detect a pressure applied to the boot main body 11. The pressure sensor 13 detects a movement of the foot of the user Us.

The conductive threads are woven at a position corresponding to a portion to which a pressure is to be applied in the foot of the user Us and which is in an area in contact with at least one selected from the group consisting of: a toe of the user Us; a sole of the user Us; a heel of the user Us; and a dorsal surface of the foot of the user Us. That is, the pressure sensor 13 is woven at the position corresponding to the portion to which a pressure is to be applied in the foot of the user Us. In Embodiment 1, as illustrated in FIG. 1, the plurality of pressure sensors 13 are woven into a sole part 10A of the room boot 10. That is, a pressure applied to the sole of the user Us is detected. The plurality of pressure sensors 13 woven into the sole part 10A each receive a different pressure applied from the sole of the user Us depending on a position into which the pressure sensor 13 is woven. Of the plurality of pressure sensors 13, pressure sensors 131 detect pressures from toes of the user Us. Of the plurality of pressure sensors 13, a pressure sensor 132 detects a pressure from a toe side of the sole of the user Us. Of the plurality of pressure sensors 13, a pressure sensor 133 detects a pressure from the heel of the user Us.

According to the above configuration, each of the pressure sensors 13 is woven into the room boot 10 at a position to which a pressure is to be applied in the foot of the user Us. This makes it possible to more specifically detect a change in pressure to the foot of the user Us. Therefore, it is possible to detect a more realistic movement of a lower body of the user Us.

The transmitting section 12 transmits, to an external device, an output signal indicating changes in pressure detected by the pressure sensors 13. The transmitting section 12 has an interface for communicating with the external device. The transmitting section 12 is detachably attached to a calf side of the boot main body 11.

According to the above configuration, it is possible to separate the transmitting section 12 and the boot main body 11 from each other. Thus, when the room boot 10 gets dirty, it is possible to wash only the boot main body 11. This makes it possible to reduce uncomfortableness that the user Us feels when wearing the room boot 10.

### Embodiment 2

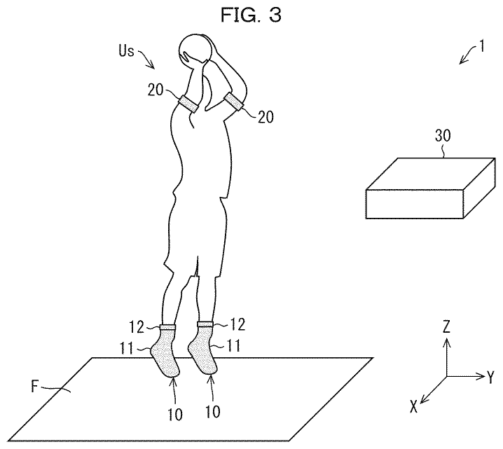

The following will describe another embodiment of the present disclosure with reference to FIGS. 2 and 3. FIG. 2 is a block diagram illustrating a configuration of an action determination system 1. FIG. 3 is a view schematically illustrating the action determination system 1 illustrated in FIG. 2. Embodiment 2 shows an example of the action determination system 1 configured to determine an action of a whole body of the user Us.

The following description assumes that coordinate axes having three directions, which are an x axis, a y axis, and a z axis are set, as illustrated in FIG. 2. Here, the z axis is an axis parallel to a direction orthogonal to a floor surface on which the user Us stands, the x axis is an axis orthogonal to the z axis, and the y axis is an axis orthogonal to the x axis and the y axis.

[Outline of Action Determination System]

As illustrated in FIGS. 2 and 3, the action determination system 1 includes a room boot(s) 10, an armband 20, and a controller 30. The armband 20 and the room boot(s) 10 are each configured to be able to perform wireless communications with the controller 30. Note that the armband 20 may be configured to perform wired communications with the controller 30.

<Room Boot>

The room boot 10 includes a first processor 14, a communication IF 15, and a pressure sensor 13. The first processor 14, the communication IF 15, and the pressure sensor 13 are connected with each other via a bus 17.

Examples of the first processor 14 include a central processing unit (CPU) and a graphic processing unit (GPU). The second processor 21 described later is configured in the same manner as the first processor 14. The first processor 14 transmits an output signal indicating a change in pressure detected by the pressure sensor 13 to the controller via the communication IF 15.

The communication IF 15 is an interface configured to perform wireless communications with the controller 30. The wireless communications may be performed in conformity to a known standard such as Bluetooth (registered trademark), an infrared ray communication, or a wireless LAN. Note that the communication IF 15 may perform communications with use of a variety of known networks. Alternatively, the communication IF 15 may perform wired communications with the controller 30. A communication IF 22 described later is configured in the same manner as the communication IF 15.

<Armband>

The armband 20 is worn around an upper limb of the user Us and configured to detect a movement of the upper limb of the user Us. The armband 20 includes a second processor 21, a communication IF 22, and an acceleration sensor 23. The second processor 21, the communication IF 22, and the acceleration sensor 23 are connected with each other via a bus 24.

The acceleration sensor 23 is a sensor configured to detect accelerations in three axial directions of the x axis, the y axis, and the z axis. The movement of the upper limb of the user Us is detected on the basis of an acceleration detected by the acceleration sensor 23. The second processor 21 transmits an output signal indicating a change in acceleration detected by the acceleration sensor 23 to the controller 30 via the communication IF 22.

<Controller>

The controller 30 is a device configured to receive output signals from: the pressure sensor(s) 13 of the room boot(s) 10; and the acceleration sensor 23 of the armband 20 and comprehensively determine an action of the whole body of the user Us on the basis of the output signals.

The controller 30 is implemented by, for example, a personal computer (PC) or a programmable logic controller (PLC). The controller 30 includes an interface configured to communicate with, for example, a processor such as a CPU or a GPU, a memory such as a read only memory (ROM) or a random access memory (RAM), and other devices.

[Flow of Operation of Action Determination System]

The controller 30 totally determines an action of the whole body of the user Us on the basis of a change(s) in pressure detected by the pressure sensor(s) 13 of the room boot(s) 10 and a change in acceleration detected by the acceleration sensor 23 of the armband 20. More specifically, the controller 30 determines a kind of the action (such as a bend of the upper limb) of an upper body of the user Us on the basis of a change in acceleration detected by the acceleration sensor 23. In Embodiment 2, the controller 30 determines, through the armband 20, an action of the upper limb of the user Us, such as a shot and a pass.

In addition, the controller 30 determines a kind of an action (such as a bend of a lower limb) of the lower limb of the user Us on the basis of a change(s) in pressure detected by the pressure sensor(s) 13. The controller 30 determines an action of the user Us and a strength of the action of the user Us through the pressure sensor(s) 13 woven into a sole part(s) 10A of the room boot(s) 10. Examples of the action of the user Us include a walk, a run, a jump, and a step-in. A strength of the action means a running ability, a jumping ability or the like. Note that, in the present disclosure, the “determine the action” includes determinations of (i) a kind of the action, (ii) accuracy of the action, and (iii) a strength of the action.

According to the above configuration, it is possible to more specifically determine an action of the whole body of the user Us on the basis of a combination of: a change in acceleration of the upper limb detected by the acceleration sensor 23 of the armband 20 and a change(s) in pressure to the lower limb detected by the pressure sensor(s) 13 of the room boot(s) 10. This makes it possible to determine a more realistic action of a whole body.

### Another Embodiment

Note that an action determination system in accordance with the present disclosure is not limited to the one relating to the game as described in the embodiments above. The action determination system in accordance with the present disclosure may be used in order to determine, in another field, an action of a whole body, which is a combination of an action of an upper body and an action of a lower body of a human or an animal. Examples of such a field include sport, care, and medicine (particularly, rehabilitation).

In the above-described embodiments, a room boot 10 for indoor use is described. However, the present disclosure is not limited to this. Examples of the footwear in accordance with the present disclosure include a shoe and a sock. Note that the footwear is not limited to one for indoor use and may be one for outdoor use. Further, the footwear in accordance with the present disclosure may be configured such that part of the lower limb of the user Us is externally exposed.

In the above-described embodiments, the position of the pressure sensor 13 woven into the room boot 10 is limited. However, the position of the pressure sensor 13 is not limited to this. In the room boot 10, the pressure sensor 13 may be disposed at a position corresponding to a dorsal surface of the foot of the user Us. The position at which the pressure sensor 13 is disposed can be changed as appropriate depending on the intended use of the footwear.

### Supplementary Note

The present disclosure is not limited to the embodiments above, but can be altered by a skilled person in the art within the scope of the claims. The present disclosure also encompasses, in its technical scope, any embodiment derived by combining technical means disclosed in differing embodiments as appropriate.

### REFERENCE SIGNS LIST

1 Action determination system
10 Room boot
20 Armband
30 Controller

1. Footwear comprising a capacitive sensor, wherein:
conductive threads are woven into a fabric of the footwear; and
the conductive threads serve as the capacitive sensor configured to detect, on a basis of a change in capacitance between the conductive threads adjacent to each other, a change in pressure applied to an area into which the conductive threads are woven.

2. The footwear according to claim 1, wherein the conductive threads are woven into a position corresponding to a portion to which a pressure is to be applied in a foot of a user and which is in an area in contact with at least one selected from the group consisting of: a toe of the user; a sole of the user; a heel of the user; and a dorsal surface of the foot of the user.

3. The footwear according to claim 1, further comprising a transmitting section configured to transmit, to an external device, data indicating a change in pressure detected by the capacitive sensor,
the transmitting section being detachably attached to the footwear.

4. An action determination system comprising:
the footwear according to claim 1;
an acceleration sensor worn around an upper limb of a user and configured to detect a movement of the upper limb of the user; and
a controller configured to execute a determination process of determining an action of a whole body of the user on a basis of a change in pressure detected by the capacitive sensor of the footwear and a change in acceleration of the upper limb of the user detected by the acceleration sensor.

5. An action determination system comprising:
the footwear according to claim 2;
an acceleration sensor worn around an upper limb of a user and configured to detect a movement of the upper limb of the user; and
a controller configured to execute a determination process of determining an action of a whole body of the user on a basis of a change in pressure detected by the capacitive sensor of the footwear and a change in acceleration of the upper limb of the user detected by the acceleration sensor.

6. An action determination system comprising:
the footwear according to claim 3;
an acceleration sensor worn around an upper limb of a user and configured to detect a movement of the upper limb of the user; and
a controller configured to execute a determination process of determining an action of a whole body of the user on a basis of a change in pressure detected by the capacitive sensor of the footwear and a change in acceleration of the upper limb of the user detected by the acceleration sensor.
